SEQUENCE LISTING (36 sequences)

SEQ ID NO: 1 - DNA, 24 bp, Escherichia coli
aaaccggctttgtcagatatgatg

SEQ ID NO: 2 - DNA, 44 bp, Escherichia coli
tgtgctcagattctgggtctcctgtgctcagattctgggtctcc

SEQ ID NO: 3 - DNA, 20 bp, Escherichia coli
tcacctttcgctcaggatgc

SEQ ID NO: 4 - DNA, 21 bp, Escherichia coli
atagcacccggtacaagcagg

SEQ ID NO: 5 - DNA, 23 bp, Escherichia coli
tttcgctcaggatgctaaaccag

SEQ ID NO: 6 - DNA, 28 bp, Escherichia coli
caggattacaacacaattcacagcagta

SEQ ID NO: 7 - DNA, 27 bp, Escherichia coli
ctttcccctcttttagtcagtcaactg

SEQ ID NO: 8 - DNA, 26 bp, Escherichia coli
caggattacaacaaagttcacagcag

SEQ ID NO: 9 - DNA, 22 bp, Escherichia coli
ggtcagcgttttttccttcctg

SEQ ID NO: 10 - DNA, 22 bp, Escherichia coli
ggccagcgttttttccttcctg

SEQ ID NO: 11 - DNA, 21 bp, Escherichia coli
tcgtcaccaaaggaatcggag

SEQ ID NO: 12 - DNA, 21 bp, Escherichia coli
tcgtcaccagaggaatcggag

SEQ ID NO: 13 - DNA, 23 bp, Escherichia coli
gtttgcagttgatgtcagaggga

SEQ ID NO: 14 - DNA, 22 bp, Escherichia coli
caacgaatggcgatttatctgc

SEQ ID NO: 15 - DNA, 23 bp, Escherichia coli
ggaatgcaaatcagtcgtcactc

SEQ ID NO: 16 - DNA, 22 bp, Escherichia coli
gcctgtcgccagttatctgaca

SEQ ID NO: 17 - DNA, 20 bp, Escherichia coli
ttgaccgcctttccgatacc

SEQ ID NO: 18 - DNA, 20 bp, Escherichia coli
atccgcatcaccgctcagac

SEQ ID NO: 19 - DNA, 21 bp, Escherichia coli
gggaaaagccggaacagttct

SEQ ID NO: 20 - DNA, 22 bp, Escherichia coli
ccagcataacagccgatgtgat

SEQ ID NO: 21 - DNA, 22 bp, Escherichia coli
tccaataagtcgcagaatgcta

SEQ ID NO: 22 - DNA, 22 bp, Escherichia coli
tccaataaggcgcagaatgcta

SEQ ID NO: 23 - DNA, 21 bp, Escherichia coli
caccgtagcctttcgctgaag

SEQ ID NO: 24 - DNA, 20 bp, Escherichia coli
ggaggcagcagtggggaata

SEQ ID NO: 25 - DNA, 20 bp, Escherichia coli
tgacgggcggtgtgtacaag

SEQ ID NO: 26 - DNA, 29 bp, Escherichia coli
ctggattcatcatgcaccacaaggttgtg

SEQ ID NO: 27 - DNA, 35 bp, Escherichia coli
cacagcagtaattgctactattcatgctttcagga

SEQ ID NO: 28 - DNA, 25 bp, Escherichia coli
gtcctgaaagcatgaatagtagcaa

SEQ ID NO: 29 - DNA, 26 bp, Escherichia coli
gagactaaaaagtgtgatgttgtaaa

SEQ ID NO: 30 - DNA, 31 bp, Escherichia coli
tacccgtttaggtattggtggcgaatactgg

SEQ ID NO: 31 - DNA, 31 bp, Escherichia coli
tccagaggaagggcggtttaataatctacgg

SEQ ID NO: 32 - DNA, 34 bp, Escherichia coli
tggtttcatcatatctggcgttaatggagttcag

SEQ ID NO: 33 - DNA, 31 bp, Escherichia coli
ccagcatctcatacttctgctcttctgcctg

SEQ ID NO: 34 - DNA, 28 bp, Escherichia coli
tgctgagaaaacaacgggaaggagagga

SEQ ID NO: 35 - DNA, 30 bp, Escherichia coli
tcagaagtaatgagcgcaacgtctgcaatt

SEQ ID NO: 36 - DNA, 29 bp, Escherichia coli
aacgtattcaccgtggcattctgatccac

(19) United States
(12) Patent Application Publication
(10) Pub. No.: US 2006/0194206 A1
(43) Pub. Date: Aug. 31, 2006

(54) Diagnostics of diarrheagenic escherichia coli (dec) and shigella spp

(76) Inventors: Soren Persson, Volosvej (DK); Flemming Scheutz, Sigerstedgade (DK)

Correspondence Address: HOWSON AND HOWSON, SUITE 210, 501 OFFICE CENTER DRIVE, FT WASHINGTON, PA, 19034, US

(21) Appl. No.: 10/564,441

(22) PCT Filed: Jul. 9, 2004
(86) PCT No.: PCT/DK04/00494

§ 371 Date: Mar. 1, 2006

(30) Foreign Application Priority Data
Jul. 14, 2003 (DK) PA 2003 01070

(51) Int. Cl. C12Q 1/68

(52) U.S. Cl. 435/6

(57) ABSTRACT

A method for the identification of the diarrheagenic E. coli groups: ETEC (enterotoxigenic E. coli), A/EEC (attaching and effacing E. coli) EPEC (enteropathogenic E. coli), VTEC (verocytotoxin producing E. coli) and EIEC (enteroinvasive E. coli), and Shigella spp. is described. The bacterial identification is made possible by the specific detection of the following virulence genes: sta and elt encoding heat stable enterotoxin (ST) and heat labile enterotoxin (LT) characteristic of ETEC, eae encoding intimin, characteristic of A/EEC, EPEC or VTEC, bfpA encoding bundle forming pilus (BfpA), characteristic of EPEC, vtx1 and vtx2 encoding veroxytotoxin 1 and 2 (VT1 and 2) characteristic of VTEC, ipah encoding invasive plasmid antigen H (IpaH) characteristic of EIEC and Shigella spp., and ehxA encoding enterohemolysin (EhxA) characteristic of some EPEC and VTEC strains. The method allows the simultaneous detection of any combination of the 8 virulence genes by one single multiplex-PCR. The method is thoroughly validated with respect to sensitivity and specificity, and showed high performance compared to other publication. The method includes an internal positive PCR control and the carry-over prevention system, UNG, which makes it ideal for routine diagnostic analyses. The method can be combined with a number of other technologies leading to even higher sensitivity and reduced time of analysis—both important parameters when diarrheagenic patient or contaminated foods are analyzed.

### FIELD OF INVENTION

The present invention relates to a novel diagnostic assay for the detection of diarrheagenic E. coli (DEC) by identification of specific genetic markers, e.g. by use of multiplex PCR. The method further allows the evaluation of the pathogenic potential, which is valuable in relation to the treatment of a patient. The method will be useful for the analysis of any material from where alive bacteria can be generated, or from where bacterial DNA can be extracted. The specific PCR product can be detected by a number of technologies that are faster and both more sensitive and specific than conventional electrophoresis. The invention also includes a method for the subtyping of a number the E. coli virulence genes that are believed to be important in the treatment and epidemiological surveillance of diarrheagenic E. coli infections.

### GENERAL BACKGROUND

Diarrheagenic E. coli (DEC) strains isolated from intestinal diseases have been grouped into at least six different categories based on epidemiological evidence, phenotypic traits, clinical features of the disease they produce, and specific virulence factors. The currently recognized categories of diarrheagenic E. coli include: Attaching and effacing E. coli (A/EEC) including Enteropathogenic E. coli (EPEC), enterotoxigenic E. coli (ETEC), Enteroinvasive E. coli (EIEC), Enteroaggregative E. coli (EAggEC), diffusely adherent E. coli (DAEC), and Shiga toxin-producing E. coli (STEC), which are also referred to as Verocytotoxin-producing E. coli (VTEC).

TABLE 1
Showing groups of diarrheagenic E. coli
and characteristic virulence genes.

| E. coli | Positive | Corresponding | |
|---|---|---|---|
| group | for gene(s) | toxin | Comments |
| VTEC | vtx1 and/ | VT1 and/or VT2 | May contain eae and/or ehxA |
| | or vtx2 | | |
| A/EEC | eae | Eae | Negative for any toxin genes |
| | | | and not belonging to the |
| | | | classical EPEC O:H serotypes. |
| EPEC | eae | Eae | Belong to classical O:H sero- |
| | | | types. Typical EPEC O:H sero- |
| | | | types are bfpA-positive, a- |
| | | | typical EPEC are bfpA-negative. |
| | | | VTEC related EPEC strains may |
| | | | contain ehxA. |
| ETEC | sta and/ | ST and/or LT | |
| | or elt | | |
| EIEC | ipaH | IpaH | |

The most important groups are EPEC, ETEC, EIEC and VTEC whereas the role of EAggEC and DAEC are still being questioned. The definitions of these groups are not definitive and related to a number of genotypic- and phenotypic methods of characterization.

A definition adopted in 1995 identified the most important characteristics of EPEC as its ability to cause attaching and effacing (A/E) histopathology and its inability to produce Verocytotoxins. Typical EPEC of human origin possess a virulence plasmid known as the EAF (EPEC adherence factor) plasmid that encodes localized adherence on cultured epithelial cells mediated by the Bundle Forming Pilus (BFP), while atypical EPEC do not posses this plasmid. The majority of typical EPEC strains fall into certain well-recognized O:H serotypes (10). According to this definition, the basic difference between typical and atypical EPEC is the presence of the EAF plasmid encoding BFP in the first group of organisms and its absence in the second. The definition is not static and may be changed as new types are discovered and described. The EPEC O:H serotypes that are currently regarded as classical and newly recognised EPEC O:H serotypes by The International Escherichia and Klebsiella Centre (WHO) are shown in table 2.

TABLE 2
O:H serotypes regarded as classical and
newly recognised EPEC O:H serotypes

| O | | |
|---|---|---|
| group | H antigen ^{a)} | Comments |
| O26 | H⁻; H11 | O26:H⁻ and O26:H11 may also be |
| | | STEC/VTEC |
| O55 | H⁻; H6; H7 | O55:H7, H10 and H⁻ may also be |
| | | STEC/VTEC |
| O86 | H⁻; H 8; H34 | 086:H− may also be EAggEC |
| | | H8 is a new type |
| O88 | H−; H25 | New type |
| O103 | H2 | |
| O111 | H⁻; H2; H7 | O111:H⁻ may also be STEC/VTEC or |
| | | EaggEC |
| O114 | H⁻; H2 | |
| O119 | H⁻; H2; H6 | |
| O125ac | H⁻; H6 | O125 may also be EaggEC |
| O126 | H⁻; H2; H21; H27 | |
| O127 | H⁻; H6; H9; H21; H40 | |
| O128ab | H⁻; H2; H7; H12 | O128:H2 may also be STEC/VTEC |
| O142 | H⁻; H6; H34 | |
| O145 | H−; H45 | New type |
| O157 | H−; H8; H16; H45 | New types |
| O158 | H⁻; H23 | |

^{a)} Non motile strains of E. coli are regarded as descendants of motile strains that have lost their motility by mutation(s).

Apart from the well-recognized classical O:H EPEC serotypes, a large group of non-classical A/EEC serotypes of E. coli strains are found to be positive for the eae-gene. Together with EPEC, this group is referred to as Attaching and Effacing E. coli (A/EEC) based on the presence of the eae-gene and absence of toxin- or invasion genes. Like EPEC, they may be positive or negative for the EAF plasmid but they may also be positive for the ehxA plasmid found in many VTEC strains, see below.

ETEC strains do not invade epithelial cells but produce one or more enterotoxins that are either heat-labile (LT), which is closely related to cholera toxin, or heat-stable (ST).

EIEC are very similar to Shigella. Like Shigella, they are capable of invading and multiplying in the intestinal epithelial cells of the distal large bowel in humans. Genes involved in the invasive phenotype of EIEC and most Shigella spp. are carried on a 140 MDa plasmid designated pInv. Prominent among these virulence genes is a type III secretion system (18). Also characteristic for the invasive phenotype is the ipaH gene, which is present in several copies on both the chromosome and the plasmid, making it especially suited as a diagnostic marker for EIEC and Shigella spp. (27).

VTEC strains are characterized by their ability to produce either one or both of at least two antigenetically distinct, usually bacteriophage-mediated cytotoxins referred to as Stx1 or VT1 (first described as Shiga-like toxin I, SLTI) and Stx2 or VT2 (first described as Shiga-like toxin II, SLTII). Whereas STEC/VTEC refers to all E. coli strains that produce Stx/VT in culture supernatants (14,15), the term enterohemorrhagic E. coli (EHEC) has been used to refer to strains that have the same clinical and pathogenic features associated with the prototype organism E. coli O157:H7 (16). In practice, EHEC is used to describe a subgroup of STEC/VTEC that causes hemorrhagic colitis (HC). Almost all STEC/VTEC O157:H7 strains harbour a large 60-65 MDa plasmid (9), designated pO157, which plays a role in the virulence (11). The large plasmid of O157 encodes the EHEC-hemolysin (Ehx), which is homologous to the E. coli α-hemolysin (20,21). A role for Ehx in the pathogenesis of diarrhoeal disease has not been demonstrated but ehxA positive VTEC strains have been found more often in patients with Hemolytic Uremic Syndrome (HUS) than in patients with diarrhoea (6) and, together with the eae-gene in VTEC strains, serve as a predictor for more serious complications. O26:H11 strains also possess at least one plasmid in the range of 55-70 MDa and other O:H serotypes show a notable similarity with the large plasmids in O157 and O26 strains (16).

The diagnosis of DEC began in 1945 when Bray demonstrated the relation between Bacterium coli var. neapolitanum and diarrhoea in humans (3). A few years later Bray and Beaven used slide agglutination to type 95% of the bacteria isolated from stool cultures from children with diarrhoea (4). The breakthrough in typing was achieved in 1950, when the E. coli serotyping scheme was developed by Kauffmann (12). During the 1950s several new serogroups were added to the list of those epidemiologically incriminated as causing diarrhoea (30). Meanwhile the enterotoxins of enterotoxigenic E. coli (ETEC) and the invasive properties of enteroinvasive E. coli (EIEC) had been described. Methods for detection included an infant mouse assay for the detection of ST, cell assays for LT, and inoculation of the eye of Guinea Pigs and subsequent development of keratoconjunctivitis for the detection of EIEC. In 1977, Konowalchuk et al. (15) discovered a cytopathic effect in Vero cells from culture filtrates of E coli. The effect could only be seen in Vero cells and not in Y1 mouse adrenal cells and Chinese hamster ovary (CHO) cells, and it was distinctly different from that of heat-labile enterotoxin. The cytotoxic effect was caused by one or more cytotoxins referred to as Vero toxins (VT) or Verocytotoxins (13).

Because of the above mentioned type diversity, rapid and easy methods of moderate cost for reliable identification and isolation of DEC strains independent of their serotype are required. A number of suitable methods for this purpose have been developed for each of the types but there is no internationally recognised standard procedure. These methods include biological assays, immunological methods, nucleic acid based assays or phenotypical tests such as O grouping of commonly occurring DEC serotypes, enterohaemolysin production of the majority of VTEC types or the failure to ferment sorbitol or produce β-glucuronidase by most VTEC O157, and culture methods.

Unfortunately, these screening methods are incomplete because they are only directed against a subset of the DEC strains.

The diagnosis of DEC have important implications for the evaluation of possible intervention during the course of illness. Prolonged diarrhoea caused by EPEC and A/EEC especially in children may require antibiotic treatment of the patient whereas treatment of patients with a VTEC infection is not recommended due to the possible increased risk of a more severe outcome. In many countries, patients with a VTEC infection are quarantined or otherwise isolated due to the risk of contaminating other people. ETEC diarrhoea is not a very serious disease and usually self-limiting. It therefore usually does not require treatment. As is the case for VTEC infections, EIEC and Shigella infections are often succeeded by both quarantine and antibiotic treatment due to the low infectious dose and the risk of contamination other people.

Pass, M. A. et al. (2001) (19) have published a method for detection of pathogenic E. coli in cultured faeces. PCR was used to amplify specific fragments in the genes encoding the following 11 virulence factors: VT1, VT2, VT2e, CNF1, CNF2, LTI, STI, STII, EaeA, Einv and Eagg. It is stated that 4 multiplex-PCR combinations of primers gave adequate amplification of their respective genes. However, when the combination of multiplex-PCR with VT1, VT2, VT2e, EaeA, CNF2, Einv, LT and ST is shown, VT2e and VT1 are not visualised on the gel. This is an accepted fact that is explained in the article. Furthermore, the assay does not include a positive control.

Patent Application WO9848046 describes a PCR assay that detects EHEC, ETEC, EPEC, EIEC and EAggEC that are specifically designed for real-time PCR analyses. However, real-time PCR is presently limited to 4 simultaneous genes per reaction because of the fluorophore overlap.

López-Saucedo, C et al (2003) (17) are describing a method where the following 7 genes are detected in the same multiplex-PCR: elt, sta, bfpA, eaeA, vtx1, vtx2 and ial. They are analysing the PCR products by size identification on agarose gel electrophoresis; the assay does not include the ehxA gene and does not have a positive control.

Compared to Prior Art the Present Method Contains the Following Advantages:

the use of ipaH for the detection of EIEC and Shigella spp. is a good genetic marker for this group of bacteria, as the gene is present in several copies both on the pInv plasmid and on the chromosome. The use of ial is a poor diagnostic marker for these bacteria because it is only present on the plasmid, which is easily lost both in vivo and in vitro.
The use of ehxA as a diagnostic marker allows a further estimation of the pathogenic potential giving rise to serious diseases, which is not possible by any of the prior art.
The use of 16S rDNA as positive control and the UNG system makes this method suitable as a reliable method for routine diagnostics. None of the prior art contains such considerations.
The present method contains thoroughly validated tests both with respect to sensitivity and specificity (see example 1).

EPEC plasmids encoding the bfpA-gene and EHEC plasmids encoding the ehxA-gene have not been found together in the same strain and the two genes may therefore serve as useful genotypic markers for the presumptive categorization of any eae-positive E. coli as either belonging to the A/EEC-EPEC group or to the EHEC group.

The method disclosed in the present invention detects the clinically relevant DEC types simultaneously and has very few limitations. The main advantage of this invention is that subsequent to the identification of positive stool cultures, procedures for further analysis by supplementary PCR of the bacterial lysates obtained during screening are possible and could include: virulence gene subtyping by PCR followed by restriction digests or sequencing, O:H serotyping by sequencing of PCR amplified bacterial antigens, or other genotyping by for example microarray analyses. The procedure also allows for the referral of the lysate to more specialised reference laboratories, which—in times where bioterrorism is ever threatening—will be safe and easy to understand for everybody at the primary screening laboratory facility. All primers chosen in the present invention were designed to match the most conserved regions within the relevant genes. By doing so, the method is optimised to detect any possible subtype of the relevant genes, including new genetic subtypes that are expected to contain genetic changes in the less conserved regions, increasing the chance of being detected by the present method. However, as for any PCR based method, it requires continuously updating and validation whenever new genotypes are being described. As our laboratory serves as The International Escherichia and Klebsiella Centre (WHO) there will be no problem in obtaining presumptive new types.

### SUMMARY OF THE INVENTION

The presently preferred embodiments of the present invention are outlined in the following points:
1) Novel multiplex-PCR combination detecting the 8 genes bfpA, ehxA, vtx1, vtx2, eaeA, ipaH, sta and elt, which is found to be the most suited gene combination for the characterization of diarrheagenic E. coli., and including a PCR-control derived from 16S rDNA (positive control).
2) Intensive validated experimental procedure, showing superior sensitivity and specificity compared to other publications.
3) Descriptions of how the multiplex-PCR can be combined with other technologies in order to decrease time of analysis and improve sensitivity.
4) Routine diagnostic consideration with respect to carry-over prevention, by the use of the UNG system.
5) Protocols for the subtyping of the virulence genes: eae, vt1 and vt2 by either, direct sequencing of the amplicons generated in the multiplex-PCR, or by sequencing of a larger fragment generated by a new PCR.

### DETAILED DESCRIPTION OF THE INVENTION

The present invention discloses a method for simultaneous detection of diarrheagenic Shigella spp. and E. coli (DEC) including A/EEC & EPEC, ETEC, VTEC, EIEC and especially strains with the ehxA gene.

A preferred embodiment of the invention detects the presence of the genes ehxA, eae, vtx1, vtx2, ipaH, sta, elt and bfpA and is able to detect Shigella spp. by the presence of the ipaH gene. The presence of the genes can be detection of the genes themselves or parts hereof, RNA or polypeptides coded by the genes.

The screening method can be performed by nucleotide sequence amplification technique, such as PCR, multiplex PCR, real-time PCR, most preferably multiplex PCR, with a selected set of primers and incorporating a positive control using 16S rDNA. Possible contamination of samples is preferably reduced by incorporating the UNG system.

Detection of the genes can be performed by size identification, e.g. by agarose gel electrophoresis or capillary electrophoresis or with a hybridisation probe.

The sample material to be analysed can be any material from where bacteria can be extracted, e.g. stool samples, consumables etc.

The screening method can be used as an in vitro diagnostic method for determining the risk of being infected with a pathogenic organism which gives rise to haemolytic uremic syndrome (HUS) or hemorrhagic colitis, by detecting the ehxA gene in the sample.

The invention discloses a specific set of primers and probes for the respective genes but are not restricted to these.

The invention also discloses a kit for the screening, which comprises, in a single or in separate containers, nucleotide sequences which are able to prime amplification in a nucleotide sequence amplification reaction, such as PCR, of the genes: ipaH, eae, ehxA, and sta, or parts of these genes or the complementary strands to the genes or parts thereof. Additionally the kit can comprise nucleotide sequences which are able to prime amplification of the genes: vtx1, vtx2, elt, and bfpA, or parts of these genes or the complementary strands to the genes or parts thereof. The described kit can also contain nucleotide sequences which are able to hybridise (preferably under stringent conditions) with the genes: ipaH, eae, ehxA, sta, vtx1, vtx2, elt, and bfpA or parts of these genes or the complementary strands to the genes or parts thereof. Preferably the kit comprises a means for a control, such as primers for 16S rDNA.

Definitions and Abbreviations:

A/E: attaching and effacing

A/EEC: attaching and effacing E. coli

Amplicon: syn. “PCR product”

bfpA: bundle forming pilus, structural gene, subunit A; Virulence factor in EPEC, which is involved in the initial adherence of the bacteria to the intestinal cells.

bp: base pair

Capillary electrophoresis: Capillary electrophoresis (CE), is a technique where an electrophoretic separation takes place in a thin capillary tube filled with buffer. A sample is injected at one end, either by electrophoresis or by pressure, and an electric field of 100 to 700 volts/centimeter is applied across the capillary. It is generally used for separating ions, which move at different speeds depending on their size and charge, when the voltage is applied. At the end of the capillary each of the separated analytes are measured by a detector in a time dependent manner. CE is usually run with an internal standard that allows size determination of the separated sample molecules.

Carry-over prevention: dUTP is incorporated in all PCR products instead of dTTP. Before PCRs are subjected to thermocycling, they are incubated with UNG (uracil-DNA glycosylase) that degrades any single or double stranded DNA containing dUTP, but has no effect on dTTP containing DNA. By this procedure, possible contamination from other PCRs is reduced, while the amplification of bacterial DNA is unaffected.

DAEC: diffusely adherent E. coli

DEC: diarrheagenic E. coli

eae: E. coli attaching and effacing: intimin. Virulence factor from EPEC, A/EEC or VTEC.

EAF: EPEC Adherence Factor plasmid. Plasmid containing BFP

EaggEC: enteroaggregative E. coli, syn. EAEC

EHEC: enterohemorrhagic E. coli

ehxA: enterohemolysin, structural gene, subunit A

EIEC: enteroinvasive E. coli

elt: gene encoding heat labile enterotoxin (LT)

EPEC: enteropathogenic E. coli

ETEC: enterotoxigenic E. coli

estA: alternative name for sta; the gene encoding heat stable enterotoxin (ST)

estA-human: the humane variant of estA (sta)

estA-porcine: the porcine variant of estA (sta)

GI: gastrointestinal tract

HC: hemorrhagic colitis

HUS: haemolytic uremic syndrom

ipaH: invasive plasmid antigen H

LT: heat-labile enterotoxin

Luminex technology: microbeads of different internal colors are labeled with hybridization probes representing different genes. These beads are hybridized with fluorescence labeled sample DNA (usually PCR products) under stringent condition. After the hybridization has taken place, the mixture is injected into the instrument that uses microfluidics to align the microbeads in a single file where lasers illuminate the colors inside and on the surface of each microbead. The optics capture the combination of color coded microbeads and hybridized sample molecule.

Microarray technology: hybridisation probes representing different genes are chemically linked to different spots on a solid surface, usually a small glass slide. Fluorescence labeled sample DNA (usually PCR products) are hybridised to the capture probes under stringent condition. After the post hybridization washing steps, only sample DNA with nucleotide sequences complementary to the capture probes will stay bound to the slide. Bound PCR products at specific spots of known capture probes, are registered by their fluorophore emission.

Multiplex PCR: PCR with more that one primer set present in the same reaction, where each primer set is amplifying a unique locus if the specific template is present.

O:H: specific serotype; “O” refers to the LPS O antigen, and H refers to flagellar antigen.

Real-time PCR: Detection of the PCR while the temperature cycling is still in progress. This can be done by the measurement of emitted fluorescence, which is linked to the reaction. The fluorescence can originated from fluororphores that bind to the double stranded amplicons (sequence unspecific interchelating agent, ex; SYBR Green), or it can originate from sequence specific probes that are designed downstream of the primers. Such probes will emit light only when digested by the polymernase because they contain a fluorophore and a corresponding quencher. Real-time PCR is limited to 4 simultanous genes per reaction because of the fluorophore overlap.

stx 1/2: genes encoding verocytotoxin 1/2

ST: heat stable enterotoxin

sta: gene encoding heat stable enterotoxin (ST); today also called estA

STh: heat stable entertoxin (human), syn. STIB

STp: heat stable enterotoxin (porcine), syn. STIA

VT 1/2: verocytotoxin 1/2, syn. shiga like toxin (Stx)

VTEC: verocytotoxin producing E. coli

vtx 1/2: gene encoding verocytotoxin 1/2 (VT 1/2)

UNG: uracil-DNA glycosylase

Enclosed in the present invention is the possibility of performing diagnostic PCR, both on DNA prepared directly from human faecal samples, or on DNA prepared from colonies grown from faecal samples. Performing PCR on DNA purified directly from faeces is an attractive strategy, as it saves time and labour. Besides that, direct PCR can detect dead cells, and cells prone to loose plasmids during in vitro growth, and is not affected by the selectivity that a growth step might introduce. However, it is also important to have a fast and easy way to test plated out cell material, when single colonies need to be isolated. For that reason, the present invention contains a method, which relies on simple boiling and centrifugation for preparing PCR-usable template DNA.

In the present invention, sensitivity limits are established by making a dilution series of pathogenic bacteria in a background of non-pathogenic bacteria. That is the most important way to test the sensitivity, as this situation is closest to the compositions of clinical samples. In the present invention, a positive result can be obtained, if the template DNA has a composition, where one pathogenic bacterium is present among 10⁴ non-pathogenic bacteria.

High specificity relies on good primer design, but is also depended on experimentally testing of the assay on different strains know to harbour different homologous of the relevant genes. The present invention has been tested 100% specific on 124 different reference strains obtained from The International Esherichia and Klebiella Centre, WHO, Statens Serum Institut, Denmark (see table 5).

In a presently preferred embodiment, the present invention is using the genes coding for the following virulence factors: BfpA, EhxA, VT1, VT2, Eae, IpaH (same as Einv), ST and LT. Genes encoding both VT2, VT2c, VT2d and subtype VT2e will be amplified by the present PCR, and result in a PCR product of the same size. Besides that, the present invention has included a universal primer-pair towards 16S rDNA as a positive control for the PCR. As for the combination of genes in the multiplex-reaction, the present invention is able to perform sensitive and specific PCR with all 8 virulence genes and 16S rDNA present in the same multiplex reaction.

The present invention includes all the relevant genes for the currently recognised and clinically important groups of DEC. The choice of genes allows for a rapid evaluation of the further treatment and interventional strategies in relation to the individual patient in order to minimise complications and the spread of highly pathogenic bacteria to contacts or the environment.

The present invention contains a method for the subtyping of the virulence factors VT1, VT2, Eae and other genes. Subtyping of these virulence genes is increasingly being accepted as an important part of characterizing VTEC infections, especially in relation to the proper treatment (5).

The present invention solves the diagnostic problem of screening for human pathogenic E. coli groups. The method relies on specific multiplex-PCR amplification of 8 virulence genes allowing a distinction between the pathogenic E. coli groups: ETEC, VTEC, A/EEC including EPEC and EIEC, and provides important distinction between typical/atypical EPEC strains and additional information on the presence or absence of the EHEC plasmid. The method is based on primers chosen to match all clinically relevant subtypes of the given virulence genes. The PCR setup is designed to enclose all primer sets in one single reaction, leading to the specific amplification of any given template present. The method was optimised to result in the best sensitivity and specificity. This was done by analysing DNA from 10-fold serial dilutions of bacterial colonies known to harbour different subtypes of the relevant virulence genes. The method therefore, allows for the analysis of any material from where alive colonies can be cultured. Of special interest is, the analysis of stool samples from diseased patients, where time, sensitivity and specificity are critical parameters in effective diagnoses. Also, the analysis of consumables has a high value as it may prohibit the spread and intake of contaminated foods. Due to the well characterized sensitivity limits, the methods also has the potential of analysing DNA purified directly from the primary sources.

The PCR primers, -probes, -reagents and -temperature conditions were optimised to perform well in combination with a number of different technologies. These technologies fall into two different groups
DNA purification directly from the source. This can be done by a number of different commercial kits described in section 5 (Example 3-6 and 9).
PCR products detection by either capillary electrophoresis, real time PCR or solid-face capture probe techniques like; membrane/ELISA hybridisation, DNA chips or Luminex®.

The present invention also encloses a real-time PCR setup with optimised PCR conditions including specific primer and probe design. Besides that, the present invention also contains the option of amplifying all 9 genes in the same reaction, in a both sensitive and specific manner (non-real-time PCR setup). This is possible because the concentration of every reagent has been carefully optimised (Example 8), and because the present invention (in this setup) is not burdened by the addition of a specific probe for every gene in the assay.

The present invention includes hybridisation probes specifically designed and optimised for constituting the capture probes, in solid surface hybridisations like membrane hybridisation or hybridisation in microtiter plates, DNA microarrays and hybridisation on microbeads (ex. Luminex® technology). Finally, the PCR products of the present invention lie within the size-range that should be easily detectable by capillary electrophoresis, which is faster, more sensitive and accurate than gel-electrophoresis.

Being able to use these technologies in combination with the multiplex PCR, results in a number of advantages compared to traditional diagnostics. Firstly, direct DNA purification from the source is not affected by the selectivity that a growth step might introduce, dead bacteria and bacteria that easily loose plasmids can be detected, and the entire procedure is much faster. Secondly, due to the multiplex setup, it only requires one PCR to screen for the entire 8 virulence genes. Thirdly, the technologies used for amplicon detection are faster and more sensitive than traditional methods.

Specific primers are of major importance in a diagnostic PCR setup. The pivot of this problem is sequence analysis of the available data in Gene Bank. The primer and probes were designed on basis of the considerations described below.

The heat stable enterotoxin (ST) of ETEC is a small monomeric protein of 18-19 amino acids encoded by the sta gene, of approximately 220 bp (18). The relative few submitted nucleotide sequences of sta, available in Gene Bank, fall into a number of phylogenetic groups based on ClustalW comparisons. The groups of genes encoding heat stable enterotoxins were as follows: five staI genes referred to as staI-human (accession numbers: J03311, M34916, M29255, M18346 and M18345) made up their own cluster, two other staI genes referred to as staI-porcine (accession numbers: M25607 and M58746) were more related to the heat stable enterotoxins from Yersinia enterocolitica and Vibrio cholera, and two separate clusters were made up of staII genes and the gene encoding EAST1 from enteroaggregative E. coli (EAggEC). As both staI-human and staI-porcine have been found in humans (18) both variants were included in the PCR. But due the sequence diversity, separate primer pairs were designed for each variant, resulting in indistinguishable product size when analysed by agarose gel electrophoresis (151 and 160 bp). Primers were not designed towards the gene encoding STII or STb (accession number M35729 and AY028790) as these variants are rarely found in humans (18). staI primers did not align with Yersinia enterocolitica heat stable enterotoxin (D63578), Vibrio cholerae heat-stable enterotoxin (M97591) or, EAST1 (AB042005), though they share some sequences similarities.

The heat labile enterotoxin (LT) of ETEC is composed of one A-subunit and 5 identical B-subunits encoded by the elt gene. The toxin can be divided into LTI and LTII based on serology and host pathogenesis. The genes encoding the A- and B-subunits are 777 bp and 375 bp long, respectively (7). The cholera enterotoxin produced by Vibrio cholerae is about 75% identical in the nucleotide sequence to the LTI of E. coli. The sequences of subunit A from nine eltI genes (accession number: V00275, S60731, AF242417, AB011677, M35581, M15261, K01995, M15362 and M57244) were compared to a number of eltII and Vibrio cholerae ctx genes. Due to the desired specificity towards E. coli, the clinical unimportance of eltII (18) and the relative low homology between eltI and eltII/ctx, probe and primers were designed to match eltI only, and result in an amplicon of 479 bp.

Intimin is encoded by the eae gene in either A/EEC (including EPEC) or VTEC, and has a size of approximately 2810 bp. Based on the divergent sequences in the last third of the 3-prime end, at least 8 subtypes can be identified. At least one of each subtype was present in the gene alignment and the following accession numbers were used: AF081186, AF253560, U60002, AB040740, AJ308552, AF116899, AF449419, AF081184, AJ308551, AF449416 and AJ298279. Probe and primes were deigned to match all tested gene sequences, and the PCR-product has a size of 377 bp.

The virulence factor, bundle forming pilus (Bfp) from EPEC, is encoded by an operon consisting of 14 genes, including the 580 bp structural gene bfpA. Based on sequence comparisons, the bfpA genes fall into an alpha and a beta type. Probe and primers were deigned to match both types by aligning the genes with the following accession number: AF304478, AF304486, AF304482, AB024946, AF304480, AF304477, AF304484, Z12295, AF382948. The resulting amplicon was 307 bp long.

Both vero toxin 1 and 2 (VT1 and VT2) from VTEC are composed of an A- and a 5 B-subunits. The gene encoding the A-subunit is approximately 960 bp long, and the gene encoding the B-subunit is approximately 270 bp long. As the homology between VT1 and VT2 is relatively low (about 50% identity), and because of the desirable differentiation between the two toxins, specific probes and primers were designed to each gene.

Based on the nucleotide sequence it is difficult to distinguish between vtx1 from E. coli and shiga toxin 1 from Shigella spp. Also, all vtx1-genes from E. coli share very high homology. Probe and primers were designed to match all vtx1-genes from E. coli and all shiga toxin 1 genes from Shigella spp, by alignment of the genes with the accession numbers: AF461172, AJ279086, AF153317, AJ132761, Z36899, AB030485, AB035142, AF461166, AJ251325, AJ314839 and M19473. The resulting PCR product is 260 bp long.

Most vtx2-genes share relative high identity (above 90%). However, one group of genes seems to make a unique cluster, consisting of the vtx2f (accession numbers AJ270998 and Aj010730) and vtx2va (M29153) (now renamed vtx2j) subtypes, with about 60% identity to the other vtx2 genes. Due the relative low sequence homology, and the fact that most vtx2f and vtx2va are not found in humans (5), probe and primers were designed to match the major vtx2 group, by aligning the vtx2 sequences: AJ313015, AP000363, AB048228, L11078, X81415, X81418, X61283, M36727, AB017524, AF291819 and Y10775. The PCR product for VT2 was designed to be 420 bp long.

Enterohemolysin A (Ehx), often found in EHEC is encoded by the ca. 3000 bp long ehxA gene, which is part of the 4-gene enterohemolysin operon. The ehxA genes are a very homogenous group of genes, and probe and primers were designed to match all known subtypes. The following accession numbers were used in the gene alignment: X79839, AB032930, AF074613, X86087, X94129, AB01549 and AF043471. The PCR product for ehxA was designed to be 530 bp long.

The invasive plasmid antigen ipaH-gene is carried in multiple copies on both the 140 MDa invasive plasmid as well as on the chromosome of EIEC strains and Shigella spp. The advantage of using this gene, rather than the ial-gene is that it remains detectable despite the loss of the plasmid. Probe and primers were designed to match all genes analysed in the gene alignment, made up of the following genes: AL391753, M32063, AF047365, M76445, M76443, AF386526, AF348706 and M76444. The size of the PCR product is 647 bp.

16S rDNA was chosen as a positive control, because many Gram-negative bacteria found in the human GI share high sequence homology. Thus, the detection of templates, that matches the primers and probe is very high under any given clinical conditions (even in antibiotic treated patients). Probe and primers were chosen to match as many as possible of the common bacteria from the human GI. The 16S rDNA control band is 1062 bp long.

Primers were designed with the following parameters: 55-57° C. melting temperature, GC-content between 45-60%, length between 20-24 bp, lowest possible likelihood of dimer- and hairpin formation, optimal entropy in the ends and distinguishable amplicon sizes. Each primer set was tested individually under varying PCR conditions, and the optimum conditions were used to construct the PCR conditions in the multiplex reaction. Primers were redesigned until they met the desired level of sensitivity and specificity so all primer sets were able to amplify any given target present in the sample. Probes were designed to have a melting temperature between 65° C. and 67° C., the least possibility of dimer- and hairpin formation and no G in the 5′-end. The primer sets were optimised by testing the PCR methods on a number of different strains under different template conditions.

The present invention also contains a method for subtyping the vtx1, vtx2 and the eae genes. By using one of the PCR primers in a sequencing reaction, performed on the PCR products, the resulting sequence can by phylogenetically analysed and assigned to a specific subtype by the comparison to sequences of known subtype. If the PCR product does not contain subtype specific sequence, a set of PCR primers can be designed to amplify a larger fragment containing more subtype specific sequences.

TABLE 3

Listed are the 8 virulence genes and 16S rDNA used in the
multiplex-PCR assay, together with amplicon size, final
primer concentration and preimer sequences.

| | | | | | | |
|---|---|---|---|---|---|---|
| Virulence | E. coli | Gene | Primer- | Amplicon | Primer conc. | Primer sequence |
| factor | group | locus | name | size (bp) | (μM) | (5′→3′) |

| | | | | | | |
|---|---|---|---|---|---|---|
| Heat labile | ETEC | elt | LT-F | 479 | 0.4 | AAACCGGCTTTG- |
| entero-toxin I | | | | | | TCAGATATGATG |
| (LT1) | | | LT-R | | 0.4 | TGTGCTCAGAT- |
| | | | | | | TCTGGGTCTCC |
| Heat stable | ETEC | sta | ST-F | 171 | 0.25 | TCACCTTTCG- |
| entero-toxin I | | | | | | CTCAGGATGC |
| (ST1) | | | ST-R | | 0.25 | ATAGCACCCG- |
| | | | | | | GTACAAGCAGG |
| | | estA- | ST-Fh | 151 | 0.4 | TTTCGCTCAGGA |
| | | humane | | | | TGCTAAACCAG |
| | | | ST-Rh | | 0.4 | CAGGATTACAACA |
| | | | | | | CAATTCACAGCAGTA |
| | | estA- | ST-Fp | 160 | 0.4 | CTTTCCCCTCTTTTAGT |
| | | porcine | | | | CAGTCAACTG |
| | | | ST-Rp | | 0.4 | CAGGATTACAACAAAG |
| | | | | | | TTCACAGCAG |
| Intimin | EPEC/ | eae | eae-F | 377 | 0.2 | GGYCAGCGTT- |
| (Eae) | VTEC | | | | | TTTTCCTTCCTG |
| | | | eae-R | | 0.2 | TCGTCACCAR- |
| | | | | | | AGGAATCGGAG |
| Shiga toxin 1 | VTEC | stx1/ | VT1-F | 260 | 0.25 | GTTTGCAGTTG- |
| (Stx1)/ | Shigella | vtx1 | | | | ATGTCAGAGGGA |
| Verocytotoxin 1 | | | VT1-R | | 0.25 | CAACGAATGG- |
| (VT1) | | | | | | CGATTTATCTGC |
| Shiga toxin 2 | VTEC | stx2/ | VT2-F | 420 | 0.25 | GGAATGCAAATC- |
| (Stx 2)/ | | vxt2 | | | | AGTCGTCACTC |
| Verocytotoxin 2 | | | VT2-R | | 0.25 | GCCTGTCGCCA- |
| (VT2) | | | | | | GTTATCTGACA |
| Invasion | EIEC/ | ipaH | ipaH-F | 647 | 0.1 | TTGACCGCCT- |
| plasmid | Shigella | | | | | TTCCGATACC |
| antigen H | | | ipaH-R | | 0.1 | ATCCGCATCA |
| (ipaH) | | | | | | CCGCTCAGAC |
| Entero- | VTEC/ | ehxA | ehx-F | 533 | 0.05 | GGGAAAAGCC- |
| hemolysin | EPEC | | | | | GGAACAGTTCT |
| A (EhxA) | | | ehx-R | | 0.05 | CCAGCATAAC- |
| | | | | | | AGCCGATGTGAT |
| bundle- | EPEC | bfpA | bfp-F | 307 | 0.4 | TCCAATAAGKC- |
| forming | | | | | | GCAGAATGCTA |
| pilus A (BfpA) | | | bfp-R | | 0.4 | CACCGTAGCCT- |
| | | | | | | TTCGCTGAAG |
| 16S rDNA | most | 16S | 16S-F | 1062 | 0.25 | GGAGGCAGCA- |
| | gram+ | | | | | GTGGGGAATA |
| | | | 165-R | | 0.25 | TGACGGGCGG- |
| | | | | | | TGTGTACAAG |

R = G or A, Y = C or T, K = G or T

The present invention contains PCR conditions that have been optimised to work with the carry-over prevention systems using dUTP and UNG. In order to prevent contamination from other PCRs, dUTP is incorporated into PCR products instead of dTTP. Before PCRs are subjected to thermocycling, they are treated with UNG that degrades dU-containing PCR products, but has no effect on native template DNA. The same level of sensitivity and specificity as described above is obtained when UNG and dUTP are included in the assay.

The PCR products can be analysed by size identification on agarose gel electrophoresis as every PCR-product has a unique size. Besides that, the present invention encloses a number of faster and more sensitive solutions for identification of the PCR products. For each gene, a hybridisation probe has been designed, from a conserved region within the gene. This feature adds an extra level of specificity, as the PCR product must have the right internal sequence in order to be detected. The specific hybridisation probes can be utilized in a number of different technologies. Firstly, real-time PCR can detect a positive PCR, before the thermocycling has run to completion. Besides the obvious time saving advantage, this technology is far more sensitive and specific than agarose gel electrophoresis. Real-time PCR is technically limited to a maximum of 4 genes per multiplex reaction, which means that the total of 9 genes needs to be analysed in 3 reactions. Secondly, the specific probes can constitute the capture-probes on solid surfaces like nylon membranes, ELISA-plates or DNA microarrays, where a stringent hybridisation can take place, which is subsequently analysed due to colour-coded reagents. Thirdly, capture-probes can also be situated on microbeads (ex. Luminex® technology), where the specific hybridization is analysed from the combination of colour-coded beads and colour-coded PCR-products. Finally, and not based on hybridisation, PCR-products can be identified by capillary electrophoresis, which is faster, more sensitive and accurate than gel-electrophoresis.

### FIGURE LEGENDS

FIG. 1. Two E. coli patogenic reference strains are mixed in equal volumes, and serially diluted in a constant background of a non-pathogenic E. coli strain (D2103). One pathogenic E. coli strain habours vtx1, eae, vtx2 and ehxA (strain D2164), while the other pathogenic strain (fr1368) contains ipaH. All PCRs contain a total amount of DNA corresponding to the preparation of approximately 0.05 bacterial colony.

Lane 1: only D2103,

Lane 2 only D2164 and fr1368,

Lane 3: 1/10 of D2164 and fr1368 relative to D2103,

Lane 4: 1/10² of D2164 and fr1368 relative to D2103,

Lane 5: 1/10³ of D2164 and fr1368 relative to D2103,

Lane 6: 1/10⁴ of D2164 and fr1368 relative to D2103,

Lane 7: 1/10⁵ of D2164 and fr1368 relative to D2103 and

Lane 8: 1/10⁶ of D2164 and fr1368 relative to D2103

FIG. 2. Two E. coli pathogenic reference strains are mixed in equal volumes, and serially diluted in a constant background of a non-pathogenic E. coli strain (D2103). One pathogenic E. coli strain habours bfpA and eae (strain D1826), while the other pathogenic strain contains sta and elt (strain D2168). All PCRs contain a total amount of DNA corresponding to the preparation of approximately 0.05 bacterial colony.

Lane 1: only D2103,

Lane 2 only D1826 and D2168,

Lane 3: 1/10 of D1826 and D2168 relative to D2103,

Lane 4: 1/10² of D1826 and D2168 relative to D2103,

Lane 5: 1/10³ of D1826 and D2168 relative to D2103,

Lane 6: 1/10⁴ of D1826 and D2168 relative to D2103,

Lane 7: 1/10⁵ of D1826 and D2168 relative to D2103 and

Lane 8: 1/10⁶ of D1826 and D2168 relative to D2103

FIG. 3. PCR test of 8 different strains (lane 1-8). Four strains (lane 1, 2, 4, and 6) harbouring a combination of the 6 virulence genes: sta, vtx1, eae, elt, ehxA and ipaH. Lane 3, 5, 7 and 8 were tested negative for pathogenic E. coli. Lane 9 contains 100 bp DNA marker. Marker to the left of lane 1 was removed for better visualization of gene designation, but was present when genes were assigned.

FIG. 4. PCR test of 8 different strains (lane 1-8) harbouring a combination of the 5 virulence genes: vtx1, bfpA, eae, vtx2 and ehxA. Lane 9 contains 100 bp DNA marker. Marker to the left of lane 1 was removed for better visualization of gene designation, but was present when genes were assigned.

### EXAMPLES

Examples 1-2 contains experimental data that shows how this method perform with respect sensitivity and specificity, and how is can be applied as a diagnostic tool in a routine laboratory. Examples 3-6 are intended to illustrate the invention, and describe how is can be applied in combination with other technologies. The combination of the following steps: a) DNA extraction; b) multiplex PCR and; c) method of PCR product detection, is not the restricted to the ones mentioned in the examples, but will work in any preferred combination. The specific method was first developed to diagnose human E. coli infections from the bacterial presence in stool samples, but most of the DNA purification methods will work on a variety of different starting materials. Example 7 deals exclusively with the genetic subtyping of genes encoding either VT2 and/or eae from either VTEC or EPEC infections.

Contents:

### Example 1

Template DNA prepared from plate grown cells by simple boiling procedure.
Multiplex-PCR on the genes encoding the following E. coli virulence factors: ST, LT, Eae, BfpA, VT1, VT2, EhxA and IpaH.
Identification of PCR products by gel electrophoresis.
This example shows how the multiplex-PCR method performs with respect to sensitivity and specificity.

### Example 2

DNA extraction from bacterial colonies, derived from fecal samples by growing fecal samples overnight on agar plates.
Multiplex PCR on the genes encoding the following E. coli virulence factors: ST, LT, Eae, VT1, VT2, and IpaH.
Identification of PCR products by gel electrophoresis.
This example shows how a method performs in a routine diagnostic laboratory compared to a DNA hybridisation technique.

### Example 3

DNA purified directly from feces by performing cell lysis and separation of magnetic beads that bind DNA (Kingfisher, Thermo Labsystems, Finland).
Multiplex PCR on the genes encoding the following E. coli virulence factors: ST, LT, Eae, BfpA, VT1, VT2, EhxA and IpaH.
Detection of PCR products by LUMINEX® technology.
This example describes a theoretical procedure for the above technologies.

### Example 4

DNA purified directly from feces, by separation of magnetic beads that bind bacterial cells followed by cell lysis and ethanol wash (Genpoint A.S, Norway)
Multiplex PCR on the genes encoding the following E. coli virulence factors: ST, LT, Eae, BfpA, VT1, VT2, EhxA and IpaH.
Detection of PCR product by hybridisation to solid-phase capture-probes on ex. nylon membrane, plastic surfaces or DNA chip/microarrays.
This example describes a theoretical procedure for the above technologies.

### Example 5

DNA purified directly from feces, by DNA absorption/entrapment in fibrous membranes (FTA Technology, Promega)
Multiplex PCR on the genes encoding the following E. coli virulence factors: ST, LT, Eae, BfpA, VT1, VT2, EhxA and IpaH.
Detection of PCR products by real-time PCR.
This example describes a theoretical procedure for the above technologies.

### Example 6

DNA purified directly from feces by use of cell lysis, absorption and elution of DNA from spin columns (ex. QIAamp® DNA Stool Mini Kit, QIAGEN).
Multiplex PCR on the genes encoding the following E. coli virulence factors: ST, LT, Eae, BfpA, VT1, VT2, EhxA and IpaH.
Detection of PCR products by use of capillary electrophoresis
This example describes a theoretical procedure for the above technologies.

### Example 7

Subtyping of the virulence genes vtx2 and eae, by direct sequencing of amplicons containing subtype distinguishable sequences.

### Example 8

Optimization of critical parameters in the multiplex PCR.

### Example 9

DNA purification from spiked stool samples by use of KingFisher and QIAamp DNA stool kit for the amplification in the multiplex PCR.

### Example 1

### Introduction

The present example describes the use of multiplex-PCR in the identification of the E. coli groups: ETEC, EPEC and A/EEC, VTEC and EIEC. The PCR relies on the specific multiplex-amplification of the genes encoding the following virulence factors: heat-labile enterotoxin (LT) characteristic for ETEC, heat-stable enterotoxin (ST) characteristic for ETEC, intimin (Eae) characteristic for EPEC or VTEC, bundle-forming pilus (BfpA) characteristic for EPEC, enterohemolysin (EhxA) characteristic for VTEC, vero toxin 1 and 2 (VT1 and VT2) characteristic for VTEC and invasive plasmid antigen (IpaH) characteristic for EIEC. As a positive control for the PCR a primerset for the 16S rDNA gene from most gram-positive bacteria was also included in the assay. The PCR analysis is performed on template DNA derived from bacterial colonies grown on plates. Each PCR was analyzed by agarose gel electrophoresis for the possible presence of amplicon(s) of the size that could be identified as any of the virulence markers mentioned above. The present example shows how the PCR method performs with respect to sensitivity and specificity. This includes the analysis of serially diluted reference strain and analysis of a 124 reference strains. All reference strains were also tested by a probe hybridization technique directed towards the same genes.

### Materials and Methods

Membrane Hybridisation:

Each virulence gene that were screened for were contained on a specific pBluescript clone. These clones served as templates for the labeling reaction using the PCR DIG Labeling Mix from Roche, and T3/T7 pBluescript primers or primers designed within the gene. Strains used to construct the clones were obtained from The International Esherichia and Klebiella Centre, WHO, Statens Serum Institut, Denmark and genes encoding the following factors were included in the assay: Eae (8), VT1 (28), VT2 (26), STp/ST1A (24), STh/STIB (24), LT (24) and IpaH (27).

By use of a 1 μL sterile loop, small volumes of the different colonies, that were chosen to be investigated, were transferred to individual spots on a nylon membrane (Hybond-N⁺, Amersham Biotech) that was positioned on top of an agar plate. After over night growth the nylon membrane was removed from the agar plate. The colonies on the nylon membrane were lysed, denatured and neutralized by incubating the nylon membrane in the following solutions for 10 min each: 10% SDS, 0.5 N NaOH, 1.4 M NaCl/0.5 M NaOH, 1 M Tris-HCl, pH 7.4 and 1.5 M NaCl/0.5 M Tris-HCl, pH 7.4. The membrane was then baked at 65° C. for one hour and prehybridised in 0.1×SSC and 0.1% SDS at 65° C.

The probe was denatured by boiling in hybridisation solution for 8 min (0.5% Blocking Reagent, 0.1% N-laurylsarcosine, 0.02% SDS and 5×SSC). The pre-hybridisation solution was exchanged with the hybridisation solution containing the denatured probe and incubated at 65° C. for one hour. The hybridisation solution was discharged and the membrane was washed twice in 2×SSC, 0.1% SDS for 5 min at room temp, and twice in 0.1×SSC, 0.1% SDS for 30 min at 65° C.

The hybridisation signals were developed by washing the membrane in Detection Buffer (0.1 M maleic acid, 0.15 M NaCl, 0.2 M NaOH, pH 7.5) for 2 min at room temperature, and in Blocking Buffer (1% Blocking Reagent, 0.1 M maleic acid, 0.15 M NaCl, 0.2 M NaOH, pH 7.5) at room temperature for 25 min. Next, 6 μL Anti-digoxigenin was mixed with 60 mL Blocking Buffer and added to the membrane and incubated at room temperature for 30 min. The membrane was then washed twice in Detection Buffer for 15 min each, and incubated for 2 min with Detection Buffer containing 50 mM MgCl₂.

Finally the membranes were incubated over night in the dark at room temperature in Detection Buffer containing 50 mM MgCl₂, 0.15% NBT (4-Nitro blue tetrazolium chloride, Boehringer Mannheim) and 0.1% BCIP (x-phosphate/5-Bromo-4-chloro-3-indolyl-phosphate, Bohringer Mannheim). Next day, the colour reaction is stopped, by rinsing the membrane in water, and the resulting hybridisation reactions were visualized for the individual spots.

PCR:

Relevant subtypes of each gene were downloaded from Gene Bank. Alignments (ClustalW algorithm) of gene sequences and primer design were done using the LaserGene Software DNAStar. Homologues of each gene that were included in the alignments are described above.

Primers sequences were chosen to have comparable GC content (45-60%), base pair length (20-24 base pairs) and melting temperatures (55-57° C.), optimal 3′ end and 5′ end stability, and low likelihood of hairpin loop and primer-dimer formation.

Primer-sets based on the theoretical primer design, were tested experimentally at different annealing temperature and different concentrations of PCR reagents. The resulting optimum conditions were taken into account when the primer sets were combined in the multiplex analysis. Primers were redesigned until they met the satisfactory level of sensitivity and specificity. Each primer set was chosen to result in a unique amplicon size that could be easily identified by agerose gel electrophoresis. See table 3 for primer sequences and amplicon sizes.

Genes encoding the following virulence factors were included in the assay: heat-labile enterotoxin (LT) characteristic for ETEC, heat-stable enterotoxin (ST) characteristic for ETEC, intimin (Eae) characteristic for A/EEC, EPEC or VTEC, bundle-forming pilus (BfpA) characteristic for EPEC, enterohemolysin (EhxA) characteristic for VTEC, vero toxin 1 and vero toxin 2 (VT1 and VT2) characteristic for VTEC, and invasive plasmid antigen (IpaH) characteristic for EIEC. As a positive control for the PCR, a primer set targeting 16S rDNA matching most gram-negative bacteria was also included.

PCRs of 25 μL were composed of the following reagents in the following order: 1×PCR buffer (Roche), 260 μM of each of dATP, dCTP, dGTP, and 520 μM of dUTP (GeneAmp, Applied Biosystems), primermix (se table 3 for individual final concentration), 0.25 U FastStart Taq DNA Polymerase (Roche), 0.25U UNG (Applied Biosystems), 2.6 mM MgCl₂ and 5 μL template DNA.

All 22 primer sequences and their individual final PCR concentrations are shown in table 3. DNA amplifications were performed in a MJ Research (PTC-200) thermocycler, with the following program: 50° C. for 1 min, 94° C. for 6 min, 35 cycles of [94° C. for 50 s, 57° C. for 40 s and 72° C. for 50 s] and 72° C. for 7 min.

Cell Preparations

Bacterial templates for sensitivity studies were prepared as follows. Reference strain D2164, fr1368, D1826, D2168 and D2103 were grown to medium sized colonies (1-2 mm) on agarose plates. One of each colony was transferred to 100 μL 10% Chelex (Bio-rad) and boiled for 5 min. The supernatants from the Chelex preparations were combined in the following way. Ten μL of strain fr1368 (ipah positive) and 10 μL of strain D2164 (vtx1, vtx2, eae and ehxA positive) were mixed and 10-fold serially diluted in water. Ten μL of each of these dilutions were mixed with 10 μL the D2103 (non-pathogenic strain) supernatant, from where 5 μL was used for PCR. Strains representing the other virulence genes D1826 (eae and bfpA positive) and D2168 (sta and elt positive) were prepared the same way. Bacterial templates for the specificity study, were prepared by growing reference strains on agarose plates. One of each colony was transferred to 100 μL 10% Chelex (Bio-rad) and boiled. Five μL of the supernatant was used directly in the PCR.

Detection of PCR-Products:

PCR products were identified on a standard agarose gel electrophoresis system by ethidium bromide staining. Gels were made of 1.5% agarose and applied voltage was 4.5 volts/cm.

### Results and Discussion

PCR primers were designed on basis of the sequence comparisons described in section 5 (Detailed description of the invention). The amplicons representing the different virulence genes were all of unique sizes, being easily distinguished by standard agarose gel electrophoresis (table 1). Intensive specificity and sensitivity studies are often not included in papers describing diagnostic PCR analyses (17,19) It is however, very important to test both parameters thoroughly before an experimental procedure is introduced into a diagnostic laboratory.

TABLE 4
Strains used to test the sensitivity limit of the multiplex-PCR assay.
Strain fr1368 and strain D2164 were mixed in equal volumes and 10-fold
serially diluted relative to the non-pathogenic strain D2103, thereby
testing the sensitivity limit of the ipaH, vt1, vt2, eae and ehxA genes.
Strain D2168 and stain D1826 were treated likewise in order to test
the sensitivity of sta, elt, eae and bfpA genes.

| Strain nr. | Virulence gene(s) |
|---|---|
| fr1368 | ipaH |
| D2164 | vxt1, vtx2, eae, ehxA |
| D2168 | sta, elt |
| D1826 | eae, bfpA |
| D2103 | non-pathogenic strains, no virulence genes |

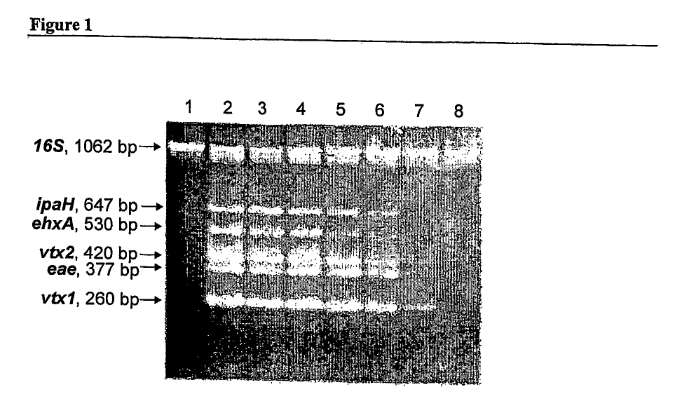

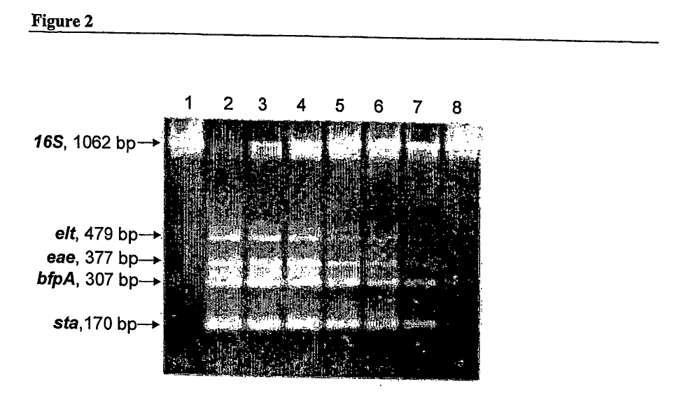

During development and optimising of the present PCR setup, a number of reference strains were tested in 10 fold serial dilutions (table 4). In order to mimic a more realistic situation 2 dilutions were made; one contains ipah, vtx1, vtx2, eae and ehxA (FIG. 1), and another dilution contains sta, elt, eae and bfpA (FIG. 2). Besides the 10-fold dilutions, the pathogenic strains were also diluted in a constant volume of a non-pathogenic E. coli strain, mimicking a situation where very few pathogenic bacteria are present in a population of non-pathogenic bacteria. This is probably the most important way to test the sensitivity limit of PCR assays, as this allows the assay to be applied on a mixture of colonies grown from faecal sample of diarrheagenic patients. Moreover, this PCR could be applied on total DNA extracted from faeces, even if very few pathogenic bacteria are present. For both dilutions, specific amplicons were detected until a dilution of 1/10⁴ relative to D2103. If one medium sized colony is estimated to contain 10⁸ bacteria and there is 1/20 colony present in each PCR, this means that at a pathogenic strain dilution of 1/10⁴ this would correspond to a sensitivity limit at approximately 500 bacteria per PCR.

In order to test the specificity of the assay 124 reference strains obtained from The International Esherichia and Klebiella Centre, WHO, Statens Serum Institut, Denmark, were tested by the assay.

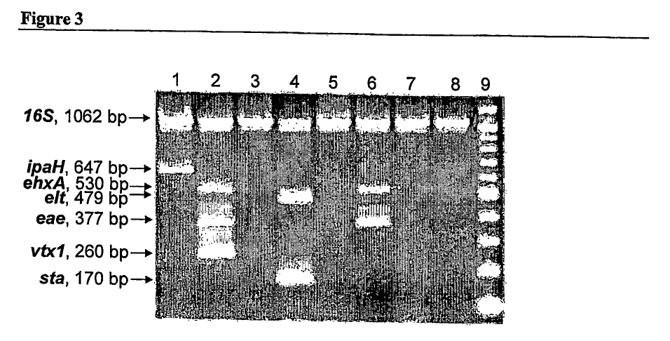

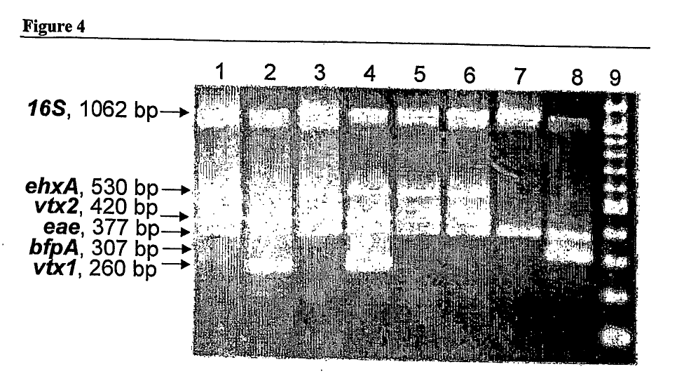

The strains were collected over a period of 19 years (1984 to 2003), and are therefore expected to constitute a broad range of relevant clinical samples representing many different homologues of the different genes (table 5). All PCR results showed the same virulence genes as also identified by standard DNA hybridisation. Example of PCR results are shown in FIGS. 3 and 4.

TABLE 5
124 reference strains obtained from the International Esherichia and Klebiella Centre, WHO, Statens
Serum Institut, Denmark. Before PCR, all strains were serotyped and tested for the 8 virulence
genes by DNA hybridisation. When tested by the multiplex-PCR method, all strains were found positive
for the exact same virulence genes as was found by hybridisation. DAEC and EAggEC were identified
by probe hybridisation for other genes not included in the PCR method.

| | | | | | | | | | | | | Negative |
|---|---|---|---|---|---|---|---|---|---|---|---|---|
| | E. coli | | | | | | | | | | | for the 8 |
| | Group/ | | | | | | | | | | | virulence |
| Nr. | Shigella | Year | Serotype | sta | vtx1 | bfpA | eae | vtx2 | elt | ehxA | ipaH | genes |
| 1 | A/EEC | 2003 | O4, 123:H− | | | | + | | | | | |
| 2 | A/EEC | 2003 | O145:H− | | | | + | | | + | | |
| 3 | A/EEC | 2003 | O116:H+ | | | | + | | | | | |
| 4 | A/EEC | 2002 | Orough:H8 | | | | + | | | | | |
| 5 | A/EEC | 2002 | O118 | | | | + | | | | | |
| 6 | A/EEC | 2002 | O35, 135:H11 | | | | + | | | | | |
| 7 | A/EEC | 2002 | O51:H49 | | | | + | | | | | |
| 8 | A/EEC | 2002 | Orough:H33 | | | | + | | | | | |
| 9 | A/EEC | 2003 | O177:H25 | | | | + | | | + | | |
| 10 | A/EEC | 2003 | O132:H34 | | | | + | | | | | |
| 10 | DAEC | 1999 | O21:H10 | | | | | | | | | + |
| 11 | DAEC | 2000 | O15:H− | | | | | | | | | + |
| 12 | DAEC | 2000 | O21:K−:H11 | | | | | | | | | + |
| 13 | DAEC | 2000 | O36:H4 | | | | | | | | | + |
| 14 | EAggEC | 2001 | O107:H+ | | | | | | | | | + |
| 15 | EAggEC | 2001 | O153:H2 | | | | | | | | | + |
| 16 | EAggEC | 2001 | O92:H33 | | | | | | | | | + |
| 17 | EAggEC | 2001 | O103:H+ | | | | | | | | | + |
| 18 | EAggEC | 2001 | O92:H+ | | | | | | | | | + |
| 19 | EAggEC | 2001 | O150:H28 | | | | | | | | | + |
| 20 | EAggEC | 2001 | O24:H+ | | | | | | | | | + |
| 21 | EAggEC | 2001 | O49:H− | | | | + | | | + | | |
| 22 | EAggEC | 2001 | O113:H− | | | | | | | | | |
| 23 | EIEC | 2000 | O64:H− | | | | | | | | + | |
| 24 | EIEC | 2000 | O64:H− | | | | | | | | + | |
| 25 | EIEC | 2001 | O+:H− | | | | | | | | + | |
| 26 | EIEC | 2001 | O121:H− | | | | | | | | + | |
| 27 | EIEC | 2001 | O28ac:H− | | | | | | | | + | |
| 28 | EIEC | 2001 | O173:H− | | | | | | | | + | |
| 29 | EIEC | 2002 | O144:H− | | | | | | | | + | |
| 30 | EIEC | 2001 | O124:H30 | | | | | | | | + | |
| 31 | EIEC | 2002 | O143:H− | | | | | | | | + | |
| 32 | EIEC | 2002 | O173:H− | | | | | | | | + | |
| 33 | EIEC | 2002 | O+:H− | | | | | | | | + | |
| 34 | EIEC | 2002 | O28ac:H− | | | | | | | | + | |
| 35 | EPEC | 2002 | O103:H2 | | | | + | | | | | |
| 36 | EPEC | 2002 | O111:H− | | | + | + | | | | | |
| 37 | EPEC | 2002 | O 26:H− | | | | + | | | + | | |
| 38 | EPEC | 2002 | O142:H34 | | | + | + | | | | | |
| 39 | EPEC | 2002 | O129:H11 | | | | + | | | | | |
| 40 | EPEC | 2002 | O111:H38 | | | | + | | | | | |
| 41 | EPEC | 2002 | O111:H9 | | | | + | | | | | |
| 42 | EPEC | 2002 | O114:H49 | | | | + | | | | | |
| 43 | EPEC | 2002 | O145:H34 | | | | + | | | | | |
| 44 | EPEC | 2002 | O121:H19 | | | | + | | | | | |
| 45 | EPEC | 2002 | O126:H6 | | | | + | | | | | |
| 46 | EPEC | 2002 | O 26:H − | | | | + | | | + | | |
| 47 | EPEC | 2002 | O 55:H 7 | | | | + | | | | | |
| 48 | EPEC | 2002 | O125ab:H 5 | | | | + | | | | | |
| 49 | EPEC | 2002 | O127:H− | | | + | + | | | | | |
| 50 | EPEC | 2002 | O145:H− | | | | + | | | + | | |
| 51 | EPEC | 2003 | O 26:H− | | | | + | | | | | |
| 52 | EPEC | 2003 | O145:H− | | | | + | | | + | | |
| 53 | EPEC | 2003 | O157:H7 | | | | + | | | | | |
| 54 | EPEC | 2003 | O114:H− | | | | + | | | | | |
| 55 | EPEC | 2003 | O86:H8 | | | | + | | | | | |
| 56 | ETEC | 2000 | Orough:H− | | | | | | | | | + |
| 57 | ETEC | 2001 | Orough:H− | + | | | | | + | | | |
| 58 | ETEC | 2000 | O167:Hru | | | | | | + | | | |
| 59 | ETEC | 2000 | O 6:H16 | + | | | | | + | | | |
| 60 | ETEC | 1996 | O115:K?:H5 | + | | | | | | | | |
| 61 | ETEC | 1996 | O8:K+:H9 | + | | | | | | | | |
| 62 | ETEC | 1998 | Orough:H− | | | | | | + | | | |
| 63 | ETEC | 2003 | O4 | | | | | | | | | + |
| 64 | ETEC | 1996 | O78:K−H11 | + | | | | | + | | | |
| 65 | ETEC | 2002 | O148 | + | | | | | + | | | |
| 66 | ETEC | 2000 | O8:H9 | + | | | | | + | | | |
| 67 | ETEC | 2000 | O39:H12 | + | | | | | + | | | |
| 68 | ETEC | 2000 | O128ac:H+ | + | | | | | + | | | |
| 69 | ETEC | 2000 | O17:Hrough | + | | | | | + | | | |
| 70 | ETEC | 2001 | O169:H− | | | | | | + | | | |
| 71 | ETEC | 2001 | O56:H− | | | | | | + | | | |
| 72 | ETEC | 2000 | O8:H9 | | | | | | + | | | |
| 73 | VTEC | 2002 | O157:H− | | + | | + | + | | + | | |
| 74 | VTEC | 2002 | O157:H− | | + | | + | + | | + | | |
| 75 | VTEC | 2002 | O157:H− | | + | | + | + | | + | | |
| 76 | VTEC | 2002 | O157:H− | | + | | + | + | | + | | |
| 77 | VTEC | 2002 | O157:H− | | + | | + | + | | + | | |
| 78 | VTEC | 1994 | O157:K:H− | | + | | + | | | + | | |
| 79 | VTEC | 1997 | O157:H 7 | | + | | + | | | + | | |
| 80 | VTEC | 1998 | O157:H 7 | | + | | + | + | | + | | |
| 81 | VTEC | 1999 | O157:H− | | + | | + | | | + | | |
| 82 | VTEC | 2002 | O157:H 7 | | | | + | + | | + | | |
| 83 | VTEC | 2002 | O157:H 7 | | | | + | + | | + | | |
| 84 | VTEC | 2002 | O157:H 7 | | | | + | + | | + | | |
| 85 | VTEC | 2002 | O157:H 7 | | | | + | + | | + | | |
| 86 | VTEC | 2002 | O157:H− | | | | + | + | | + | | |
| 87 | VTEC | 2002 | O103:H 2 | | + | | + | | | + | | |
| 88 | VTEC | 2002 | O103:Hru | | + | | + | | | + | | |
| 89 | VTEC | 2002 | O103:H 2 | | + | | + | | | + | | |
| 90 | VTEC | 2002 | O103:H 2 | | + | | + | | | + | | |
| 91 | VTEC | 2002 | O103:H 2 | | + | | + | | | + | | |
| 92 | VTEC | 1999 | O103:H 2 | | | | + | + | | + | | |
| 93 | VTEC | 1999 | O103:H 2 | | | | + | + | | + | | |
| 94 | VTEC | 1999 | O103:H 2 | | | | + | + | | + | | |
| 95 | VTEC | 2001 | O 26:H− | | + | | + | + | | + | | |
| 96 | VTEC | 1984 | O 26:H11 | | | | + | + | | + | | |
| 97 | VTEC | 2001 | O 26:H− | | + | | + | + | | + | | |
| 98 | VTEC | 2002 | O 26:H11 | | + | | + | + | | + | | |
| 99 | VTEC | 2002 | O 26:H11 | | + | | + | | | + | | |
| 100 | VTEC | 2002 | O 26:H11 | | + | | + | | | + | | |
| 101 | VTEC | 2002 | O 26:H11 | | + | | + | | | + | | |
| 102 | VTEC | 2002 | O 26:H11 | | + | | + | | | + | | |
| 103 | VTEC | 2002 | O 26:H11 | | + | | + | | | + | | |
| 104 | VTEC | 2000 | O 26:H11 | | | | + | + | | | | |
| 105 | VTEC | 2002 | O 26:H11 | | | | + | + | | + | | |
| 106 | VTEC | 2002 | O 26:H− | | | | + | + | | + | | |
| 107 | VTEC | 2002 | O 26:H11 | | | | + | + | | + | | |
| 108 | VTEC | 2001 | O145:H+ | | + | | + | | | + | | |
| 109 | VTEC | 2001 | O145:H+ | | + | | + | | | + | | |
| 110 | VTEC | 2001 | O145:H+ | | + | | + | | | + | | |
| 111 | VTEC | 2001 | O145:H28 | | + | | + | | | + | | |
| 112 | VTEC | 2002 | O145:H28 | | + | | + | | | + | | |
| 113 | VTEC | 2002 | O145:H− | | | | + | + | | + | | |
| 114 | VTEC | 2002 | O145:H− | | | | + | + | | + | | |
| 115 | VTEC | 2002 | O145:H− | | | | + | + | | + | | |
| 116 | VTEC | 2002 | O145:H− | | | | + | + | | + | | |
| 117 | VTEC | 2002 | O145:H − | | | | + | + | | + | | |
| 118 | Shigella | 2001 | sonnei | | | | | | | | + | |
| 119 | Shigella | 2001 | flexneri | | | | | | | | + | |
| 120 | Shigella | 2001 | fleneri 1b | | | | | | | | + | |
| 121 | Shigella | 2001 | flexneri 2a | | | | | | | | + | |
| 122 | Shigella | 2001 | boydii 1-7 | | | | | | | | + | |
| 123 | Shigella | 2001 | dysenteriae 2-7 | | | | | | | | + | |
| 124 | Shigella | 2001 | nonagglutinable (2) | | | | | | | | + | |

Finally, 16 non-E. coli strains were tested by the PCR method in order to investigate any cross reaction to other species. The strains were: Salmonella enterritidis, Salmonella para A, Salmonella typhimurium, Vibrio cholera, Aeromonas caviae, Shigella flexneri 2a, Shigella dysenteri 3, Proteus, Pseudomonas, Plesiomonas shigelloides, Serratia marcescens, Shigella sonni, Klebsiella, Citrobacter freundii, Salmonella cholerasuis, Yersinia ent. biotype5, 27. Except for the 3 Shigella species that showed an ipaH band, non of the other species resulted in any signals (data not shown)

### Example 2

In this examples a less complex multiplex-PCR assay was tested on 499 of clinical samples that were also tested by DNA hybridisation. This multiplex-PCR is able to identify the same E. coli groups (VTEC, EPEC and A/EEC, ETEC or EIEC) as the method described in examples 1, but relies on fewer genes and therefore gives a less informative diagnostic answer. The PCR analysis is directed towards genes encoding the following virulence factors: heat-labile enterotoxin (LT) characteristic for ETEC, heat-stable enterotoxin (ST) characteristic for ETEC, intimin (Eae) characteristic for EPEC, A/EEC or VTEC, vero toxin 1 and 2 (VT1 and VT2) characteristic for VTEC and invasive plasmid antigen (IpaH) characteristic for EIEC. The PCR analysis is performed on template DNA derived from bacterial colonies grown from faecal samples. Each PCR was analysed by agarose gel electrophoresis for the possible presence of amplicon(s) of the size that could be identified as any of the virulence markers mentioned above. In this example, the diagnostic quality of multiplex-PCR analysis is compared to membrane hybridisation, which is a traditional method for diagnostics of pathogenic E. coli groups.

### Materials and Methods

Sample Handling:

Bacterial colonies were cultured from faecal samples as follows. A small volume (approximately 0.1 g) of the faeces sample was gently shaken in 2 mL sterile buffered saltwater (80 mM NaCl, 50 mM Na₂HPO₄, 10 mM KH₂PO₄, pH 7.38). Approximately 10 μL of that suspension was streaked out onto an agar plate (SSI Enteric Medium, Statens Serum Institut, Denmark) and grown overnight at 37° C.

Membrane Hybridisation:

As described in Example 1.

Template Preparation:

From each clinical samples a number of morphological different colonies were picked from the plate grown cells and transferred to the same 200 μL 10% Chelex (Bio-Rad) and boiled for 5 min. Five μL of supernatants were used for PCR.

PCR

The multiplex-PCR contained primer set for the following genes: elt, sta, eae, vtx1, vtx2, ipaH and 165 rDNA in concentration listed in table 3. All other parameters and reagents were the same as described in example 1.

Detection of PCR-Products:

PCR products were identified on a standard agarose gel electrophoresis system by ethidium bromide staining. Gels were made of 1.5% agarose and applied voltage was 4.5 volts/cm.

### Results and Discussion

Growing faecal samples on SSI Enteric Media allows a certain differentiation of bacterial species based on phenotypical characteristics. Different strains are however not always associated with a significant phenotype. It is therefore important to pick as many different colonies as possible from a plate, in order to increase the chance of having the possible pathogenic bacteria included. With the sensitivity limit established in example 1, a positive result can be obtained if just one of the picked colonies among 10⁴ colonies is positive.

TABLE 6
Performance of multiplex-PCR and membrane hybridisation in
faeces diagnostics of E. coli. A total of 499 samples were
analysed; both method found the same 27 samples positive and
465 samples negative. 5 samples were tested positive only
by PCR, whereas 2 samples were positive only by hybridisation.
All discrepancies were double tested by PCR.

| | Found by | Only found by | Only found by | |
|---|---|---|---|---|
| Gene(s) | both methods | multiplex-PCR | DNA-hybridisation | Total |
| Negative | 465 | | | |
| eae | 19 | 2 | 1 | |
| vtx1 | 1 | 1 | | |
| vtx2 | 1 | 1 | | |
| elt | 3 | | | |
| vtx1 + eae | 1 | | | |
| ipaH | 2 | 1 | 1 | |
| Total | 492 | 5 | 2 | 499 |

In the present example 499 clinical samples were tested by both multiplex-PCR and DNA hybridisation. The results are summarized in table 6. Both methods detected the same 465 samples as negative and 27 positive samples distributed on 6 genotypes. Five samples were found positive only by PCR, whereas 2 samples were positive only by DNA hybridisation. All discrepancies were retested by PCR and showed the same result, decreasing the likelihood of a failed PCR. One explanation of the different result could therefore be differences in the quality of the picked colonies. As different persons might have picked different colonies and as some plates might have had very few pathogenic colonies on them, some differences are expected to be present in the DNA composition used in the 2 assays. With the relative higher number of positives found by PCR and the obvious time saving advantages, the PCR method is concluded to be superior to the hybridisation technique.

### Example 3

### Introduction

Multiplex-PCR analysis for the diagnosis of the pathogenic E. coli strains; VTEC, EPEC and A/EEC, ETEC or EIEC in human faecal samples. The PCR analysis is performed on template DNA purified directly from human stool samples. The subsequent PCR analysis is directed towards genes encoding the following virulence factors: LT characteristic for ETEC, ST characteristic for ETEC, Eae characteristic for EPEC, A/EEC or VTEC, BfpA characteristic for EPEC, EhxA characteristic for VTEC, VT1 and VT2 characteristic for VTEC and IpaH characteristic for EIEC. Completed PCRs were analysed by the Lurninex® technology, where PCR products are hybridised to specific capture-probes situated on microbeads.

### Materials and Methods

DNA Preparation:

DNA isolation from faecal samples was done by using the KingFisher 96™ from ThermoLabsystem, according to the manufactures instruction. Briefly, a small volume (approximately 0.1 g) of the faeces sample was gently shaken in 2 mL sterile buffered saltwater (80 mM NaCl, 50 mM Na₂HPO₄, 10 mM KH₂PO₄, pH 7.38). 200 μL of each faecal suspension was mixed with 750 μL lysis buffer and loaded in separate wells in a 96 well microtiter plate. Other plates were prepared containing either: washing buffer, 70% ethanol, distilled water and suspensions of magnetic particles. Five μL of the final DNA concentrate was used per PCR.

Sensitivity studies were performed, by adding 10-fold serially dilutions, of strains harbouring virulence gene(s), to a faecal sample tested negative for that specific gene(s).

PCR:

PCR conditions were the same as described in Example 1, except that the forward primers were 5′-biotinylated in this analysis.

Detection of PCR-Products:

PCR products were detected by the Luminex® technology. Amplicons were labelled by having the forward primers 5′-biotinylated. Capture probes (listed in table 7) were synthesized with a 5′-amine Uni-Link modification, for the coupling to the carboxylated microbeads.

Each probe was coupled to a uniquely coloured population of carboxylated microbeads. This was done by mixing 1 nmol of amine-substituted probe with a suspension of 5×10⁶ microbeads in 50 μL 0.1 M 2-(N-Morpholino) ethanesulfonic acid, pH 4.5 [MES], followed by addition of 25 μg N-(3-Dimethylaminopropyl)-N′-ethylcarbodiimide [EDC] and incubation in the dark for 30 min. The EDC addition and incubation were repeated and the microbeads were washed once with 0.02% Tween-20 and once with 0.1% SDS. Coupled microbeads were stored in TE buffer (10 mmol/L Tris-HCl, 1 mmol/L EDTA, pH 8.0) in the refrigerator in the dark until hybridization.

Five μL of the PCRs was denatured at 95° C. for 10 min and added to the hybridization solution (3 M tretramethylammonium chloride, 50 mM Tris-HCl, pH 8.0, 4 mM EDTA, pH 8.0, 0.1% sakrosyl) containing a mixture of 5000 of each probe-coupled microbead in a 50 μL total reaction volume. Reactions were hybridized at 55° C. for 10 min, pelleted by microcentrifugation and resuspended in 50 μL hybridization solution. Hybridized amplicons were labelled with 120 ng streptavidin-R-phycoerythrin at 55° C. for 5 min (62 μL total reaction volume). Reactions were then analyzed on the Luminex® 100.

1. A screening method for simultaneous detection of diarrheagenic Shigella species and E. coli (DEC) including A/EEC & EPEC, ETEC, VTEC, EIEC and strains with the ehxA gene, wherein said method comprises:
a) detecting Shigella species by detecting the presence of the ipaH gene;
b) incorporation of a 16S rDNA positive control;
c) primers chosen to match all clinically relevant subtypes of the given virulence gene;
d) performance with multiplex PCR;
e) a PCR setup designed to enclose all primer sets in one single reaction, leading to the specific amplification of any given template present
f) primers selected from table 3;
g) use of the UNG system.

2-38. (canceled)

39. The screening method according to claim 1, detecting the genes selected from the group comprising: ipah, eae, sta, vtx1, vtx2, and et, parts of these genes or products of these genes or parts thereof, such as RNA or polypeptides.

40. The screening method according to claim 1, detecting the genes selected from the group comprising: ipaH, eae, ehxA, sta, vtx1, vtx2, elt, and bfpA, parts of these genes or products of these genes or parts thereof, such as RNA or polypeptides.

41. The screening method according to claim 1 wherein the genes are detected by size identification.

42. The screening method according to claim 41 wherein the means for detecting by size identification is performed by agarose gel electrophoresis or capillary electrophoresis.

43. The screening method according to claim 1 wherein the genes are detected with a hybridization probe.

44. The screening method according to claim 43 wherein the probes are selected from table 7.

45. The screening method according to claim 1 wherein the material to be analyzed is selected from the group consisting of stool samples, consumables, bacterial cultures, and sewage samples.

46. The screening method according to claim 45, in which the testing is carried out on a sample from a human or an animal or from food or beverages.

47. The screening method according to claim 1, in which the primers used are selected from the group consisting of:
a) the primers of table 3;
b) sequences having a sequence identity of at least 80% (such as at least 85%, at least 90%, or at least 95%) with the primer sequences of a);
c) parts of the sequences in a) or b), having a length of more than 10, preferably more than 13 nucleotides;
d) sequences comprising a sequence in a), b) or c), said sequence having a length of no more than 100 nucleotides.

48. The screening method according to claim 47 wherein said primers consist of 14, 15, 16, 17, 18, 19, 20, 21 or 22 consecutive nucleotides of the sequences in a) or b).

49. The screening method according to claim 47 wherein said primers consist of at most 90, 80, 70, 60, 50, 40, or 30 nucleotides of the sequences comprising a), b), or c).

50. The screening method according to claim 1, in which the probes used are selected from the group consisting of:
a) the probe sequences of table 7;
b) sequences having a sequence identity of at least 80% with the primer sequences of a);
c) parts of the sequences in a) or b), having a length of more than 10, preferable more than 16 nucleotides, such as more than 17, 18, 19 or 20 nucleotides;
d) sequences comprising a sequence in a), b) or c), said sequence having a length of no more than 100 nucleotides.

51. The screening method according to claim 50 wherein said probes have at least 85%, 90%, or 95% sequence identity with the sequences of a).

52. The screening method according to claim 50 wherein said probes consist of 14, 15, 16, 17, 18, 19, 20, 21 or 22 consecutive nucleotides of the sequences in a) or b).

53. The screening method according to claim 50 wherein said probe consist of at most 90, 80, 70, 60, 50, 40, or 30 nucleotides of the sequences comprising a), b), or c).

54. A kit which comprises, in a single or in separate containers, nucleotide sequences which are able to prime amplify, in a nucleotide sequence amplification reaction, the genes: ipaH, eae, sta, vtx1, vtx2, and elt or parts of these genes or the complementary strands to the genes or parts thereof and which comprises a control.

55. The kit according to claim 54 wherein the sequence amplification reaction is PCR.

56. The kit according to claim 54 wherein the control consists of primers for 16s rDNA.

57. The kit according to claim 54 wherein the nucleotide sequences for priming are selected from the group consisting of the priming sequences in table 3.

58. The kit according to claim 54 wherein the nucleotide sequences for probing are selected from the group consisting of the probe sequences in table 7.

59. The kit according to claim 54 which comprises a means for detecting by size identification.

60. The kit according to claim 59 wherein the means for detecting by size identification is performed by agarose gel electrophoresis or capillary electrophoresis.
